(19) United States
(12) Patent Application Publication
(10) Pub. No.: US 2011/0039856 A1
(43) Pub. Date: Feb. 17, 2011

(54) POLYMORPHS OF A C-MET/HGFR INHIBITOR

(75) Inventors: Benjamin Micah Collman, Wilmette, IL (US); Jingrong Jean Cui, San Diego, CA (US)

Correspondence Address: PFIZER INC, 10555 SCIENCE CENTER DRIVE, SAN DIEGO, CA, 92121, US

(73) Assignee: Pfizer Inc.

(21) Appl. No.: 12/745,411

(22) PCT Filed: Nov. 29, 2007
(86) PCT No.: PCT/IB2008/003170

§ 371 Date: Nov. 2, 2010

Related U.S. Application Data
(60) Provisional application No. 60/991,169, filed on Nov. 29, 2007.

(51) Int. Cl. A61K 31/4985; C07D 487/04; A61P 35/00; A61P 35/02

(52) U.S. Cl. 514/249; 544/350

(57) ABSTRACT

This invention relates to polymorphs of 2-[4-(3-Quinolin-6-ylmethyl-3H-[1,2,3]triazolo[4,5-b]pyrazin-5-yl)-pyrazol-1-yl]-ethanol that are useful in the treatment of abnormal cell growth, such as cancer, in mammals. This invention also relates to compositions including such salts and polymorphs, and to methods of using such compositions in the treatment of abnormal cell growth in mammals, especially humans.

This application is a 371 application of PCT/IB2008/003170, filed on Nov. 18, 2008, which claims the benefit of U.S. Provisional Application No. 60/991,169 filed on Nov. 29, 2007, the contents of which is hereby incorporated by reference in its entirety.

### FIELD OF THE INVENTION

This invention relates to salts and polymorphs of 2-[4-(3-quinolin-6-ylmethyl-3H-[1,2,3]triazolo[4,5-b]pyrazin-5-yl)-pyrazol-1-yl]-ethanol useful in the treatment of abnormal cell growth, such as cancer, in mammals. This invention also relates to compositions including such salts and polymorphs, and to methods of using such compositions in the treatment of abnormal cell growth in mammals, especially humans.

### BACKGROUND OF THE INVENTION

The compound 2-[4-(3-quinolin-6-ylmethyl-3H-[1,2,3]triazolo[4,5-b]pyrazin-5-yl)-pyrazol-1-yl]-ethanol (also herein referred to as “compound 1”) represented by the formula 1

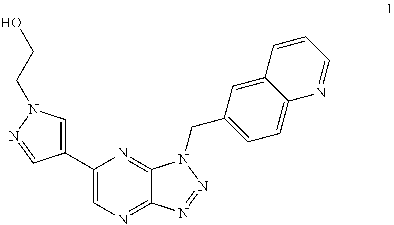

is a potent small-molecule inhibitor of c-Met/HGFR (hepatocyte growth factor receptor) kinase and ALK (anaplastic lymphoma kinase) activity. Compound 1 has anti-tumor properties that are pharmacologically mediated through inhibition of c-Met/HGFR which is involved in the regulation of growth and metastatic progression of a wide variety of tumors types, and ALK which implicated in the pathogenesis of ALCL (anaplastic large cell lymphoma). Compound 1 is disclosed in International Patent Application WO 2007/132308 and U.S. Pat. No. 7,732,604, both of which are herein incorporated by reference in their entirety.

Human cancers comprise a diverse array of diseases that collectively are one of the leading causes of death in developed countries throughout the world (American Cancer Society, Cancer Facts and Figures 2005. Atlanta: American Cancer Society; 2005). The progression of cancers is caused by a complex series of multiple genetic and molecular events including gene mutations, chromosomal translocations, and karyotypic abnormalities (Hanahan D, Weinberg R A. The hallmarks of cancer. Cell 2000; 100: 57-70). Although the underlying genetic causes of cancer are both diverse and complex, each cancer type has been observed to exhibit common traits and acquired capabilities that facilitate its progression. These acquired capabilities include dysregulated cell growth, sustained ability to recruit blood vessels (i.e., angiogenesis), and ability of tumor cells to spread locally as well as metastasize to secondary organ sites (Hanahan D, Weinberg R A. The hallmarks of cancer. Cell 2000; 100: 57-70). Therefore, the ability to identify novel therapeutic agents that 1) inhibit molecular targets that are altered during cancer progression or 2) target multiple processes that are common to cancer progression in a variety of tumors presents a significant unmet need.

Example 209 of U.S. patent application Ser. No. 11/745,921 describes the preparation of the mesylate salt of compound 1 which was found to be a crystalline polymorph. It is advantageous to have salt and polymorphic forms having improved properties, such as improved crystallinity, dissolution properties, and/or decreased hygroscopicity, while maintaining chemical and enantiomeric stability properties.

### SUMMARY OF THE INVENTION

In one embodiment, the present invention provides a compound comprising a salt selected from the group consisting of 2-[4-(3-quinolin-6-ylmethyl-3H-[1,2,3]triazolo[4,5-b]pyrazin-5-yl)-pyrazol-1-yl]-ethanol hydrochloride salt, 2-[4-(3-quinolin-6-ylmethyl-3H-[1,2,3]triazolo[4,5-b]pyrazin-5-yl)-pyrazol-1-yl]-ethanol maleate salt, 2-[4-(3-quinolin-6-ylmethyl-3H-[1,2,3]triazolo[4,5-b]pyrazin-5-yl)-pyrazol-1-yl]-ethanol phosphate salt, 2-[4-(3-quinolin-6-ylmethyl-3H-[1,2,3]triazolo[4,5-b]pyrazin-5-yl)-pyrazol-1-yl]-ethanol sulfate salt, and 2-[4-(3-quinolin-6-ylmethyl-3H-[1,2,3]triazolo[4,5-b]pyrazin-5-yl)-pyrazol-1-yl]-ethanol tosylate salt. In particular aspects of this embodiment, the salt is anhydrous. In a further aspect the salt is crystalline. In a further aspect the salt is a crystalline anhydrous salt. In a further aspect the salt is a substantially pure polymorph. In a further aspect the salt is a compound comprising 2-[4-(3-quinolin-6-ylmethyl-3H-[1,2,3]triazolo[4,5-b]pyrazin-5-yl)-pyrazol-1-yl]-ethanol hydrochloride salt. In a further aspect the salt is a compound comprising a compound comprising 2-[4-(3-quinolin-6-ylmethyl-3H-[1,2,3]triazolo[4,5-b]pyrazin-5-yl)-pyrazol-1-yl]-ethanol maleate salt. In a further aspect the salt is a compound comprising a compound comprising 2-[4-(3-quinolin-6-ylmethyl-3H-[1,2,3]triazolo[4,5-b]pyrazin-5-yl)-pyrazol-1-yl]-ethanol phosphate salt. In a further aspect the salt is a compound comprising a compound comprising 2-[4-(3-quinolin-6-ylmethyl-3H-[1,2,3]triazolo[4,5-b]pyrazin-5-yl)-pyrazol-1-yl]-ethanol sulfate salt. In a further aspect the salt is a compound comprising a compound comprising 2-[4-(3-quinolin-6-ylmethyl-3H-[1,2,3]triazolo[4,5-b]pyrazin-5-yl)-pyrazol-1-yl]-ethanol tosylate salt.

In yet another embodiment, the present invention provides a pharmaceutically acceptable salt of the compound of the formula 1

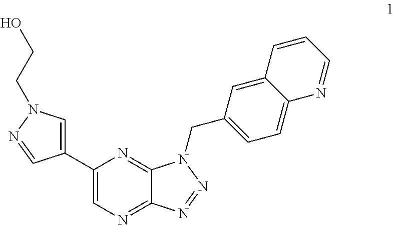

with the proviso that the pharmaceutically acceptable salt is not a mesylate salt. In a further aspect the pharmaceutically acceptable salt is crystalline. In a further aspect the pharmaceutically acceptable salt is a crystalline anhydrous salt. In a further aspect the pharmaceutically acceptable salt is a substantially pure polymorph. In a further aspect the pharmaceutically acceptable salt is a hydrochloride salt. In a further aspect the pharmaceutically acceptable salt is a maleate salt. In a further aspect the pharmaceutically acceptable salt is a phosphate salt. In a further aspect the pharmaceutically acceptable salt is a sulfate salt. In a further aspect the pharmaceutically acceptable salt is a tosylate salt.

In a further aspect, the present invention provides a compound comprising a crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising a peak at diffraction angle (2θ) of 27.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 17.7±0.2 and 27.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 17.7±0.2, 24.5±0.2, and 27.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 17.7±0.2, 24.5±0.2, 26.5±0.2, and 27.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 17.7±0.2, 24.5±0.2, 25.6±0.2, 26.5±0.2, and 27.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 11.6±0.2, 17.7±0.2, 24.5±0.2, 25.6±0.2, 26.5±0.2, and 27.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) of 11.6±0.2, 17.7±0.2, 20.3±0.2, 24.5±0.2, 25.6±0.2, 26.5±0.2, and 27.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol hydrochloride has a powder X-ray diffraction pattern comprising peaks at diffraction angles (2θ) essentially the same as shown in FIG. 1.

In a further aspect, the present invention provides a compound comprising a crystalline salt of 2-[4-(3-quinolin-6-ylmethyl-3H-[1,2,3]triazolo[4,5-b]pyrazin-5-yl)-pyrazol-1-yl]-ethanol maleate. In a further aspect, the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising a peak at diffraction angle (2θ) of 24.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 22.6±0.2 and 24.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 13.2±0.2, 22.6±0.2, and 24.6±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 12.9±0.2, 13.2±0.2, 22.6±0.2, and 24.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 12.9±0.2, 13.2±0.2, 16.6±0.2, 22.6±0.2, and 24.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 12.9±0.2, 13.2±0.2, 16.1±0.2, 16.6±0.2, 22.6±0.2, and 24.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) of 12.9±0.2, 13.2±0.2, 16.1±0.2, 16.6±0.2, 22.6±0.2, 23.9±0.2, and 24.6±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol maleate has a powder X-ray diffraction pattern comprising peaks at diffraction angles (2θ) essentially the same as shown in FIG. 2.

In a further aspect, the present invention provides a compound comprising a crystalline salt of 2-[4-(3-quinolin-6-ylmethyl-3H-[1,2,3]triazolo[4,5-b]pyrazin-5-yl)-pyrazol-1-yl]-ethanol phosphate. In a further aspect, the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising a peak at diffraction angle (2θ) of 17.0±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2 and 20.9±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, and 24.8±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, 24.8±0.2, and 25.8±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, 24.8±0.2, 25.8±0.2, and 28.4±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, 24.8±0.2, 25.8±0.2, 27.0±0.2, and 28.4±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, 24.8±0.2, 25.8±0.2, 27.0±0.2, 28.4±0.2, and 28.9±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) essentially the same as shown in FIG. 3.

In a further aspect, the present invention provides a compound comprising a crystalline salt of 2-[4-(3-quinolin-6-ylmethyl-3H-[1,2,3]triazolo[4,5-b]pyrazin-5-yl)-pyrazol-1-yl]-ethanol sulfate. In a further aspect, the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising a peak at diffraction angle (2θ) of 15.2±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2 and 18.0±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2, 18.0±0.2, and 25.0±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2, 18.0±0.2, 25.0±0.2, and 27.2±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2, 18.0±0.2, 25.0±0.2, 25.7±0.2, and 27.2±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2, 18.0±0.2, 22.0±0.2, 25.0±0.2, 25.7±0.2, and 27.2±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) of 15.2±0.2, 18.0±0.2, 22.0±0.2, 25.0±0.2, 25.7±0.2, 27.2±0.2, and 27.8±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol sulfate has a powder X-ray diffraction pattern comprising peaks at diffraction angles (2θ) essentially the same as shown in FIG. 4.

In a further aspect, the present invention provides a compound comprising a crystalline polymorph salt form of 2-[4-(3-quinolin-6-ylmethyl-3H-[1,2,3]triazolo[4,5-b]pyrazin-5-yl)-pyrazol-1-yl]-ethanol tosylate. In a further aspect, the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising a peak at diffraction angle (2θ) of 24.4±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 16.5±0.2 and 24.4±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 16.5±0.2, 17.2±0.2, and 24.4±0.2. In a further aspect, the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 14.9±0.2, 16.5±0.2, 17.2±0.2, and 24.4±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 10.7±0.2, 14.9±0.2, 16.5±0.2, 17.2±0.2, and 24.4±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 10.7±0.2, 14.9±0.2, 16.5±0.2, 17.2±0.2, 24.4±0.2, and 27.2±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) of 10.7±0.2, 14.9±0.2, 16.5±0.2, 17.2±0.2, 24.4±0.2, 26.6±0.2, and 27.2±0.2. In a further aspect the crystalline salt of 2-[4-(3-quinolin-6-ylmethyl-3H-[1,2,3]triazolo[4,5-b]pyrazin-5-yl)-pyrazol-1-yl]-ethanol tosylate has a powder X-ray diffraction pattern comprising peaks at diffraction angles (2θ) essentially the same as shown in FIG. 5.

The present invention further provides a pharmaceutical composition comprising a crystalline salt of 2-[4-(3-quinolin-6-ylmethyl-3H-[1,2,3]triazolo[4,5-b]pyrazin-5-yl)-pyrazol-1-yl]-ethanol hydrochloride. The present invention further provides a pharmaceutical composition comprising crystalline salt of 2-[4-(3-quinolin-6-ylmethyl-3H-[1,2,3]triazolo[4,5-b]pyrazin-5-yl)-pyrazol-1-yl]-ethanol maleate. The present invention further provides a pharmaceutical composition comprising crystalline salt of 2-[4-(3-quinolin-6-ylmethyl-3H-[1,2,3]triazolo[4,5-b]pyrazin-5-yl)-pyrazol-1-yl]-ethanol phosphate. The present invention further provides a pharmaceutical composition comprising crystalline salt of 2-[4-(3-quinolin-6-ylmethyl-3H-[1,2,3]triazolo[4,5-b]pyrazin-5-yl)-pyrazol-1-yl]-ethanol sulfate. The present invention further provides a pharmaceutical composition comprising crystalline salt of 2-[4-(3-quinolin-6-ylmethyl-3H-[1,2,3]triazolo[4,5-b]pyrazin-5-yl)-pyrazol-1-yl]-ethanol tosylate.

The present invention further provides a capsule comprising said pharmaceutical composition. In particular aspects of this embodiment, the capsule comprises from 0.2 to 200 mg of the crystalline salt of 2-[4-(3-quinolin-6-ylmethyl-3H-[1,2,3]triazolo[4,5-b]pyrazin-5-yl)-pyrazol-1-yl]-ethanol hydrochloride. In a further aspect the capsule comprises from 25 to 150 mg of the crystalline salt of 2-[4-(3-quinolin-6-ylmethyl-3H-[1,2,3]triazolo[4,5-b]pyrazin-5-yl)-pyrazol-1-yl]-ethanol hydrochloride. In a further embodiment, the capsule comprises from 50 to 100 mg of the crystalline salt of 2-[4-(3-quinolin-6-ylmethyl-3H-[1,2,3]triazolo[4,5-b]pyrazin-5-yl)-pyrazol-1-yl]-ethanol maleate. In particular aspects of this embodiment, the capsule comprises from 0.2 to 200 mg of the crystalline salt of 2-[4-(3-quinolin-6-ylmethyl-3H-[1,2,3]triazolo[4,5-b]pyrazin-5-yl)-pyrazol-1-yl]-ethanol maleate. In a further aspect the capsule comprises from 25 to 150 mg of the crystalline salt of 2-[4-(3-quinolin-6-ylmethyl-3H-[1,2,3]triazolo[4,5-b]pyrazin-5-yl)-pyrazol-1-yl]-ethanol maleate. In a further embodiment, the capsule comprises from 50 to 100 mg of the crystalline salt of 2-[4-(3-quinolin-6-ylmethyl-3H-[1,2,3]triazolo[4,5-b]pyrazin-5-yl)-pyrazol-1-yl]-ethanol maleate. In particular aspects of this embodiment, the capsule comprises from 0.2 to 200 mg of the crystalline salt of 2-[4-(3-quinolin-6-ylmethyl-3H-[1,2,3]triazolo[4,5-b]pyrazin-5-yl)-pyrazol-1-yl]-ethanol phosphate. In a further aspect the capsule comprises from 25 to 150 mg of the crystalline salt of 2-[4-(3-quinolin-6-ylmethyl-3H-[1,2,3]triazolo[4,5-b]pyrazin-5-yl)-pyrazol-1-yl]-ethanol phosphate. In a further embodiment, the capsule comprises from 50 to 100 mg of the crystalline salt of 2-[4-(3-quinolin-6-ylmethyl-3H-[1,2,3]triazolo[4,5-b]pyrazin-5-yl)-pyrazol-1-yl]-ethanol phosphate. In particular aspects of this embodiment, the capsule comprises from 0.2 to 200 mg of the crystalline salt of 2-[4-(3-quinolin-6-ylmethyl-3H-[1,2,3]triazolo[4,5-b]pyrazin-5-yl)-pyrazol-1-yl]-ethanol sulfate. In a further aspect the capsule comprises from 25 to 150 mg of the crystalline salt of 2-[4-(3-quinolin-6-ylmethyl-3H-[1,2,3]triazolo[4,5-b]pyrazin-5-yl)-pyrazol-1-yl]-ethanol sulfate. In a further embodiment, the capsule comprises from 50 to 100 mg of the crystalline salt of 2-[4-(3-quinolin-6-ylmethyl-3H-[1,2,3]triazolo[4,5-b]pyrazin-5-yl)-pyrazol-1-yl]-ethanol sulfate. In particular aspects of this embodiment, the capsule comprises from 0.2 to 200 mg of the crystalline salt of 2-[4-(3-quinolin-6-ylmethyl-3H-[1,2,3]triazolo[4,5-b]pyrazin-5-yl)-pyrazol-1-yl]-ethanol tosylate. In a further aspect the capsule comprises from 25 to 150 mg of the crystalline salt of 2-[4-(3-quinolin-6-ylmethyl-3H-[1,2,3]triazolo[4,5-b]pyrazin-5-yl)-pyrazol-1-yl]-ethanol tosylate. In a further embodiment, the capsule comprises from 50 to 100 mg of the crystalline salt of 2-[4-(3-quinolin-6-ylmethyl-3H-[1,2,3]triazolo[4,5-b]pyrazin-5-yl)-pyrazol-1-yl]-ethanol tosylate.

In another embodiment, the invention provides a method of treating cancer in a mammal, including a human, the method comprising administering to the mammal a therapeutically effective amount of a pharmaceutical composition of the present invention.

In another embodiment, the invention provides a method of treating cancer in a mammal, the method comprising administering to the mammal, including a human, a capsule of the present invention.

In another embodiment, the present invention provides a method of treating abnormal cell growth in a mammal, including a human, in need of such treatment comprising, administering to said mammal a therapeutically effective amount of the crystalline salt of 2-[4-(3-quinolin-6-ylmethyl-3H-[1,2,3]triazolo[4,5-b]pyrazin-5-yl)-pyrazol-1-yl]-ethanol hydrochloride. In another embodiment, the present invention provides a method of treating abnormal cell growth in a mammal, including a human, in need of such treatment comprising, administering to said mammal a therapeutically effective amount of the crystalline salt of 2-[4-(3-quinolin-6-ylmethyl-3H-[1,2,3]triazolo[4,5-b]pyrazin-5-yl)-pyrazol-1-yl]-ethanol maleate. In another embodiment, the present invention provides a method of treating abnormal cell growth in a mammal, including a human, in need of such treatment comprising, administering to said mammal a therapeutically effective amount of the crystalline salt of 2-[4-(3-quinolin-6-ylmethyl-3H-[1,2,3]triazolo[4,5-b]pyrazin-5-yl)-pyrazol-1-yl]-ethanol phosphate. In another embodiment, the present invention provides a method of treating abnormal cell growth in a mammal, including a human, in need of such treatment comprising, administering to said mammal a therapeutically effective amount of the crystalline salt of 2-[4-(3-quinolin-6-ylmethyl-3H-[1,2,3]triazolo[4,5-b]pyrazin-5-yl)-pyrazol-1-yl]-ethanol sulfate. In another embodiment, the present invention provides a method of treating abnormal cell growth in a mammal, including a human, in need of such treatment comprising, administering to said mammal a therapeutically effective amount of the crystalline salt of 2-[4-(3-quinolin-6-ylmethyl-3H-[1,2,3]triazolo[4,5-b]pyrazin-5-yl)-pyrazol-1-yl]-ethanol tosylate.

In another embodiment, the abnormal cell growth is mediated by at least one genetically altered tyrosine kinase. In another embodiment, the abnormal cell growth is mediated by hepatocyte growth factor receptor (c-Met/HGFR) kinase or anaplastic lymphoma kinase (ALK). In another embodiment, the abnormal cell growth is mediated by hepatocyte growth factor receptor (c-Met/HGFR) kinase. In another embodiment, the abnormal cell growth is mediated by anaplastic lymphoma kinase (ALK).

In another embodiment, the abnormal cell growth is cancer. In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment, the cancer is selected from the group consisting of non-small cell lung cancer (NSCLC), squamous cell carcinoma, hormone-refractory prostate cancer, papillary renal cell carcinoma, colorectal adenocarcinoma, neuroblastomas, anaplastic large cell lymphoma (ALCL) and gastric cancer.

### DEFINITIONS

As used herein, unless otherwise indicated, the term “abnormal cell growth” refers to cell growth that is independent of normal regulatory mechanisms (e.g., loss of contact inhibition).

As used herein, the term “substantially pure” with reference to a particular polymorphic or amorphous form means that the polymorphic amorphous form includes less than 10%, preferably less than 5%, preferably less than 3%, preferably less than 1% by weight of any other physical forms of the compound.

As used herein, unless otherwise indicated, the term “treating” means reversing, alleviating, or inhibiting the progress of the disorder or condition to which such term applies, or one or more symptoms of such disorder or condition. The term “treatment”, as used herein, unless otherwise indicated, refers to the act of treating as “treating” as defined immediately above.

As used herein, the term “essentially the same” with reference to X-ray diffraction peak positions means that typical peak position and intensity variability are taken into account. For example, one skilled in the art will appreciate that the peak positions (2θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only.

### BRIEF DESCRIPTION OF THE DRAWINGS

FIG. 1 shows a powder X-ray diffraction pattern of the crystalline salt of 2-[4-(3-quinolin-6-ylmethyl-3H-[1,2,3]triazolo[4,5-b]pyrazin-5-yl)-pyrazol-1-yl]-ethanol hydrochloride.

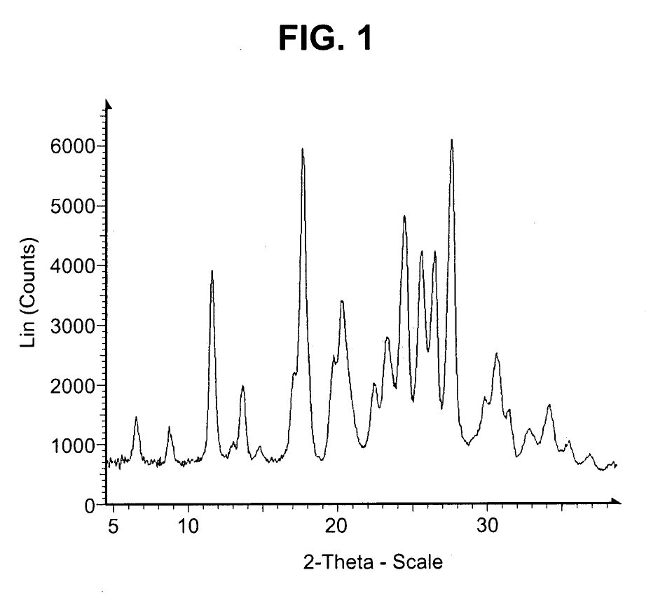

FIG. 2 shows a powder X-ray diffraction pattern of the crystalline salt of 2-[4-(3-quinolin-6-ylmethyl-3H-[1,2,3]triazolo[4,5-b]pyrazin-5-yl)-pyrazol-1-yl]-ethanol maleate.

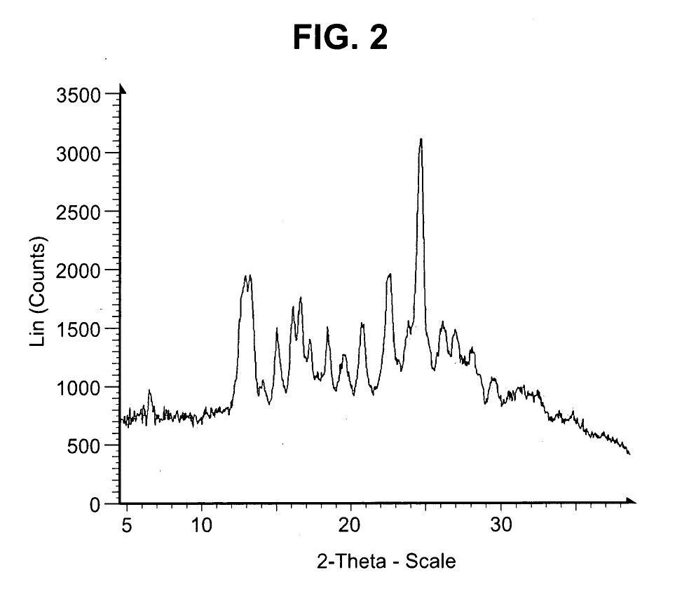

FIG. 3 shows a powder X-ray diffraction pattern of the crystalline salt of 2-[4-(3-quinolin-6-ylmethyl-3H-[1,2,3]triazolo[4,5-b]pyrazin-5-yl)-pyrazol-1-yl]-ethanol phosphate.

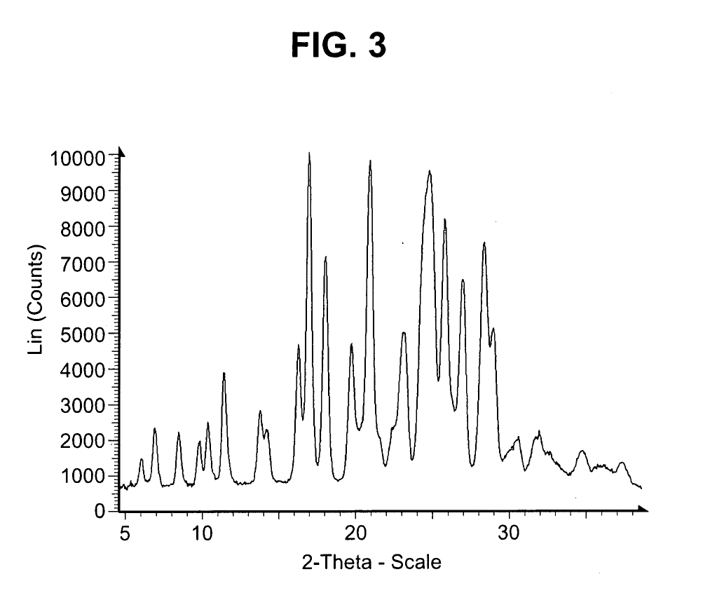

FIG. 4 shows a powder X-ray diffraction pattern of the crystalline salt of 2-[4-(3-quinolin-6-ylmethyl-3H-[1,2,3]triazolo[4,5-b]pyrazin-5-yl)-pyrazol-1-yl]-ethanol sulfate.

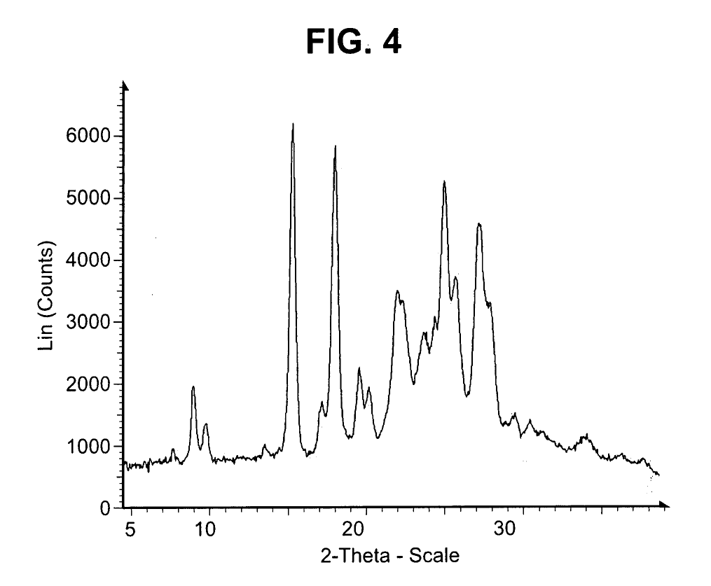

FIG. 5 shows a powder X-ray diffraction pattern of the crystalline salt of 2-[4-(3-quinolin-6-ylmethyl-3H-[1,2,3]triazolo[4,5-b]pyrazin-5-yl)-pyrazol-1-yl]-ethanol tosylate.

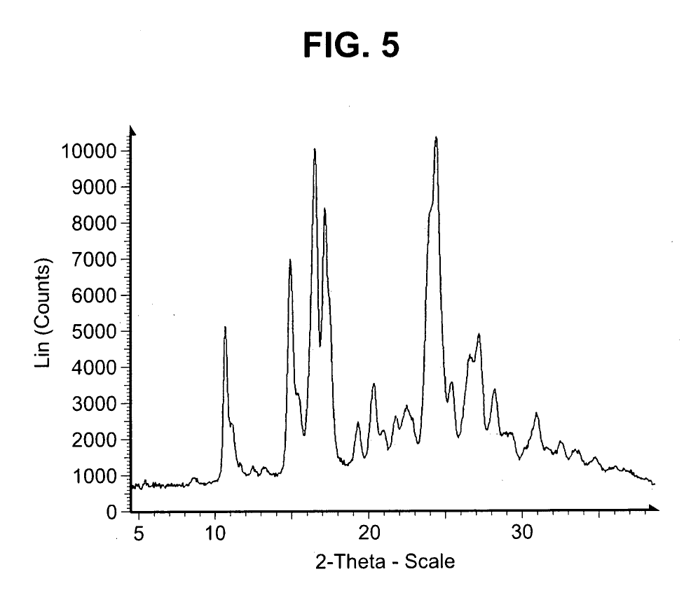

FIG. 6 shows a differential scanning calorimetery (DSC) thermogram of the crystalline salt of 2-[4-(3-quinolin-6-ylmethyl-3H-[1,2,3]triazolo[4,5-b]pyrazin-5-yl)-pyrazol-1-yl]-ethanol hydrochloride.

FIG. 7 shows a differential scanning calorimetery (DSC) thermogram of the crystalline salt of 2-[4-(3-quinolin-6-ylmethyl-3H-[1,2,3]triazolo[4,5-b]pyrazin-5-yl)-pyrazol-1-yl]-ethanol maleate.

FIG. 8 shows a differential scanning calorimetery (DSC) thermogram of the crystalline salt of 2-[4-(3-quinolin-6-ylmethyl-3H-[1,2,3]triazolo[4,5-b]pyrazin-5-yl)-pyrazol-1-yl]-ethanol phosphate.

FIG. 9 shows a differential scanning calorimetery (DSC) thermogram of the crystalline salt of 2-[4-(3-quinolin-6-ylmethyl-3H-[1,2,3]triazolo[4,5-b]pyrazin-5-yl)-pyrazol-1-yl]-ethanol sulfate.

FIG. 10 shows a differential scanning calorimetery (DSC) thermogram of the crystalline salt of 2-[4-(3-quinolin-6-ylmethyl-3H-[1,2,3]triazolo[4,5-b]pyrazin-5-yl)-pyrazol-1-yl]-ethanol tosylate.

### DETAILED DESCRIPTION OF THE INVENTION

Several unique crystalline salts of 2-[4-(3-quinolin-6-ylmethyl-3H-[1,2,3]triazolo[4,5-b]pyrazin-5-yl)-pyrazol-1-yl]-ethanol have now been made. The free base compound 2-[4-(3-quinolin-6-ylmethyl-3H-[1,2,3]triazolo[4,5-b]pyrazin-5-yl)-pyrazol-1-yl]-ethanol can be prepared according to methods described in U.S. patent application Ser. No. 11/745,921 the entire disclosure of which is incorporated herein by reference.

Salts of 2-[4-(3-quinolin-6-ylmethyl-3H-[1,2,3]triazolo[4,5-b]pyrazin-5-yl)-pyrazol-1-yl]-ethanol can be prepared by treating the free base compound with a suitable amount of the chosen mineral or organic acid in an aqueous solvent medium or in a suitable organic solvent, such as methanol, acetonitrile, ethanol, or ethyl acetate. Upon careful evaporation of the solvent, the desired solid salt is obtained. The desired acid salt can also be precipitated from a solution of the free base in an organic solvent by adding to the solution an appropriate mineral or organic acid.

### Hydrochloride Salt

The HCl salt of 2-[4-(3-quinolin-6-ylmethyl-3H-[1,2,3]triazolo[4,5-b]pyrazin-5-yl)-pyrazol-1-yl]-ethanol can be produced with good crystallinity, for example, by stirring the free base compound in any suitable solvent, including but not limited to, CH₂Cl₂, acetone, THF, acetonitrile, ethyl acetate, methanol, ethanol, water, isopropyl alcohol, or a mixture thereof, and 2M HCl at an elevated temperature (e.g. ˜68° C.), then cooling to room temperature. After cooling the solution, the resulting HCl salt in crystalline form precipitates and can be collected by filtration.

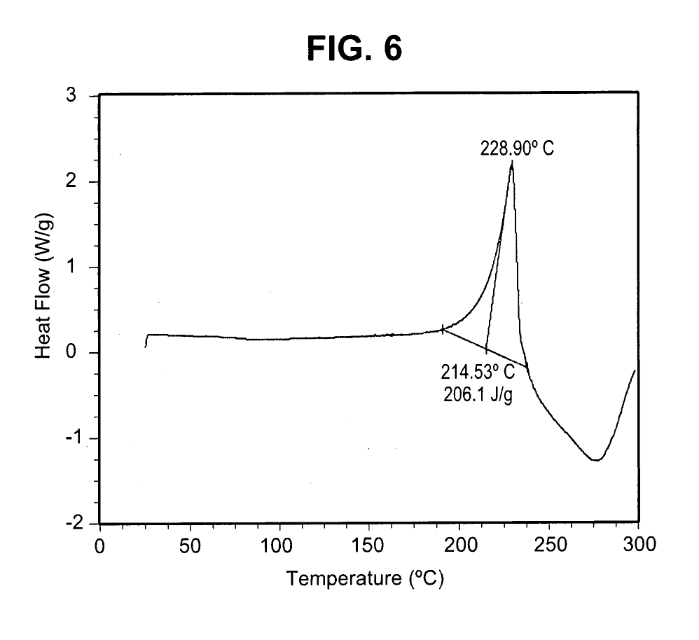

The powder X-ray diffraction (PXRD) pattern of the crystalline HCl salt of 2-[4-(3-quinolin-6-ylmethyl-3H-[1,2,3]triazolo[4,5-b]pyrazin-5-yl)-pyrazol-1-yl]-ethanol is shown in Table 1. The DSC thermogram for the HCl salt is shown in FIG. 6.

Table 1: PXRD data tabulation for the crystalline polymorph form 1 of 2-[4-(3-quinolin-6-ylmethyl-3H-[1,2,3]triazolo[4,5-b]pyrazin-5-yl)-pyrazol-1-yl]-ethanol hydrochloride (Example 1).

TABLE 1

| 2θ (°) | Intensity % |
|---|---|
| 6.5 | 24.2 |
| 8.7 | 21.3 |
| 11.6 | 64.1 |
| 13.0 | 17 |
| 13.7 | 32.6 |
| 17.1 | 36.2 |
| 17.7 | 97.7 |
| 19.8 | 40.7 |
| 20.3 | 55.9 |
| 22.5 | 33.1 |
| 23.3 | 45.8 |
| 24.5 | 79.1 |
| 25.6 | 69.3 |
| 26.5 | 69.4 |
| 27.6 | 100 |
| 29.9 | 29.2 |
| 30.6 | 41.3 |
| 31.5 | 25.9 |
| 32.8 | 20.4 |
| 34.2 | 27.1 |
| 35.5 | 17 |
| 36.9 | 13.5 |

### Maleate Salt

The maleate salt of 2-[4-(3-quinolin-6-ylmethyl-3H-[1,2,3]triazolo[4,5-b]pyrazin-5-yl)-pyrazol-1-yl]-ethanol can be produced with good crystallinity, for example, by placing maleic acid and 2-[4-(3-quinolin-6-ylmethyl-3H-[1,2,3]triazolo[4,5-b]pyrazin-5-yl)-pyrazol-1-yl]-ethanol in a vial and dissolving in any suitable solvent, including but not limited to, CH₂Cl₂, acetone, THF, acetonitrile, ethyl acetate, methanol, ethanol, water, isopropyl alcohol, or a mixture thereof, then adding a suitable cosolvent, including but not limited to, CH₂Cl₂, acetone, THF, acetonitrile, ethyl acetate, methanol, ethanol, water, isopropyl alcohol, or a mixture thereof, followed by crystallization from any suitable solvent, including but not limited to, CH₂Cl₂, acetone, THF, acetonitrile, ethyl acetate, methanol, ethanol, water, isopropyl alcohol, or a mixture thereof.

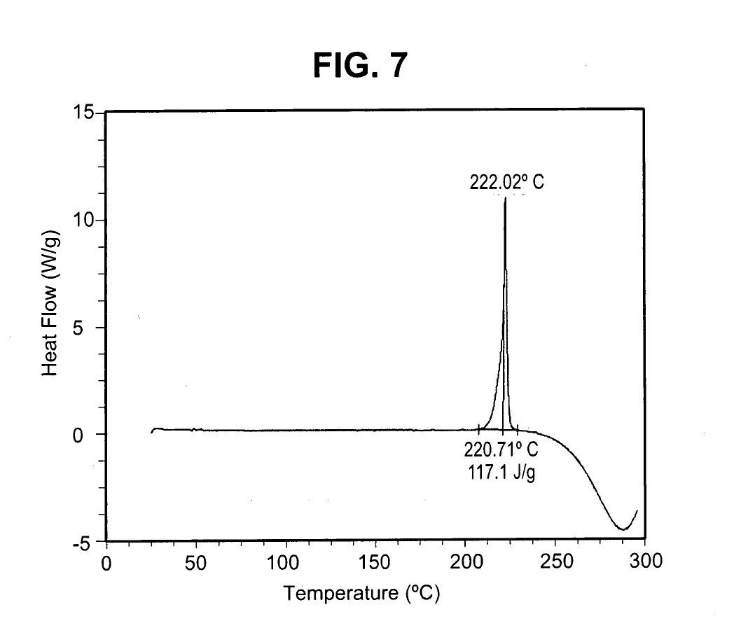

The powder X-ray diffraction (PXRD) pattern of the crystalline maleate salt of 2-[4-(3-quinolin-6-ylmethyl-3H-[1,2,3]triazolo[4,5-b]pyrazin-5-yl)-pyrazol-1-yl]-ethanol is shown in Table 2. The DSC thermogram for the maleate salt is shown in FIG. 7.

TABLE 2

| 2θ (°) | Intensity % |
|---|---|
| 6.5 | 31.4 |
| 12.9 | 62.5 |
| 13.2 | 62.8 |
| 15.0 | 48.1 |
| 16.1 | 53.9 |
| 16.6 | 56.7 |
| 17.2 | 45 |
| 18.4 | 48.4 |
| 19.6 | 40.7 |
| 20.8 | 49.5 |
| 22.6 | 62.8 |
| 23.9 | 50.1 |
| 24.6 | 100 |
| 26.1 | 49.8 |
| 27.0 | 47.7 |
| 28.1 | 43 |
| 29.6 | 34.2 |

### Phosphate Salt

The phosphate salt of 2-[4-(3-quinolin-6-ylmethyl-3H-[1,2,3]triazolo[4,5-b]pyrazin-5-yl)-pyrazol-1-yl]-ethanol can be produced with good crystallinity, for example, by stirring 2-[4-(3-quinolin-6-ylmethyl-3H-[1,2,3]triazolo[4,5-b]pyrazin-5-yl)-pyrazol-1-yl]-ethanol with H₃PO₄ in an appropriate solvent, including but not limited to, including but not limited to, CH₂Cl₂, acetone, THF, acetonitrile, ethyl acetate, methanol, ethanol, water, isopropyl alcohol, or a mixture thereof, then stirring the resulting solid in an appropriate solvent, including but not limited to, including but not limited to, CH₂Cl₂, acetone, THF, acetonitrile, ethyl acetate, methanol, ethanol, water, isopropyl alcohol, or a mixture thereof, followed by crystallization from an appropriate solvent, including but not limited to, including but not limited to, CH₂Cl₂, acetone, THF, acetonitrile, ethyl acetate, methanol, ethanol, water, isopropyl alcohol, or a mixture thereof.

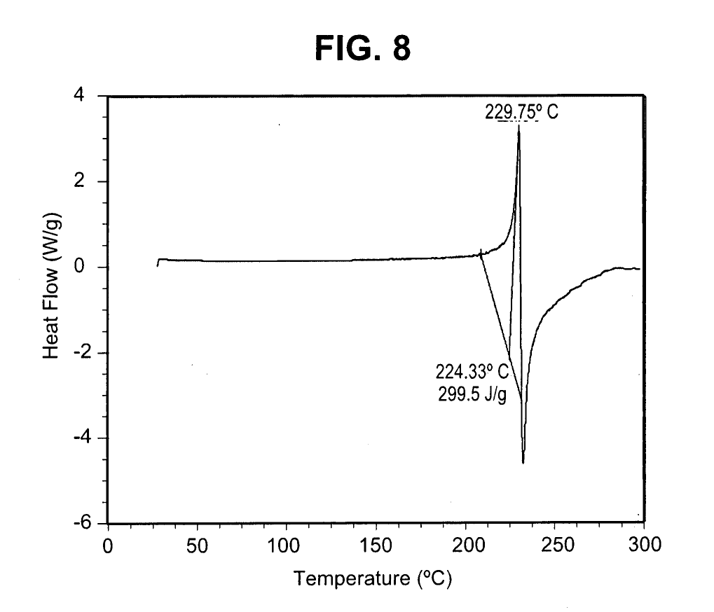

The powder X-ray diffraction (PXRD) pattern of the crystalline phosphate salt of 2-[4-(3-quinolin-6-ylmethyl-3H-[1,2,3]triazolo[4,5-b]pyrazin-5-yl)-pyrazol-1-yl]-ethanol is shown in Table 3. The DSC thermogram for the phosphate salt is shown in FIG. 8.

TABLE 3

| 2θ (°) | Intensity % |
|---|---|
| 6.0 | 14.7 |
| 6.9 | 23.4 |
| 8.4 | 22.3 |
| 9.7 | 19.4 |
| 10.3 | 25 |
| 11.4 | 38.7 |
| 13.8 | 28.2 |
| 16.3 | 46.6 |
| 17.0 | 100 |
| 18.0 | 71 |
| 19.7 | 46.9 |
| 20.9 | 97.8 |
| 23.1 | 49.8 |
| 24.8 | 94.9 |
| 25.8 | 81.5 |
| 27.0 | 64.5 |
| 28.4 | 75 |
| 28.9 | 51.1 |
| 30.6 | 19.8 |
| 31.9 | 22.3 |
| 34.8 | 16.8 |
| 37.4 | 13.3 |

### Sulfate Salt

The sulfate salt of 2-[4-(3-quinolin-6-ylmethyl-3H-[1,2,3]triazolo[4,5-b]pyrazin-5-yl)-pyrazol-1-yl]-ethanol can be produced with good crystallinity, for example, by stirring 2-[4-(3-quinolin-6-ylmethyl-3H-[1,2,3]triazolo[4,5-b]pyrazin-5-yl)-pyrazol-1-yl]-ethanol with H₂SO₄ in an appropriate solvent, including but not limited to, including but not limited to, CH₂Cl₂, acetone, THF, acetonitrile, ethyl acetate, methanol, ethanol, water, isopropyl alcohol, or a mixture thereof, then stirring the resulting solid in an appropriate solvent, including but not limited to, including but not limited to, CH₂Cl₂, acetone, THF, acetonitrile, ethyl acetate, methanol, ethanol, water, isopropyl alcohol, or a mixture thereof, followed by crystallization from an appropriate solvent, including but not limited to, including but not limited to, CH₂Cl₂, acetone, THF, acetonitrile, ethyl acetate, methanol, ethanol, water, isopropyl alcohol, or a mixture thereof.

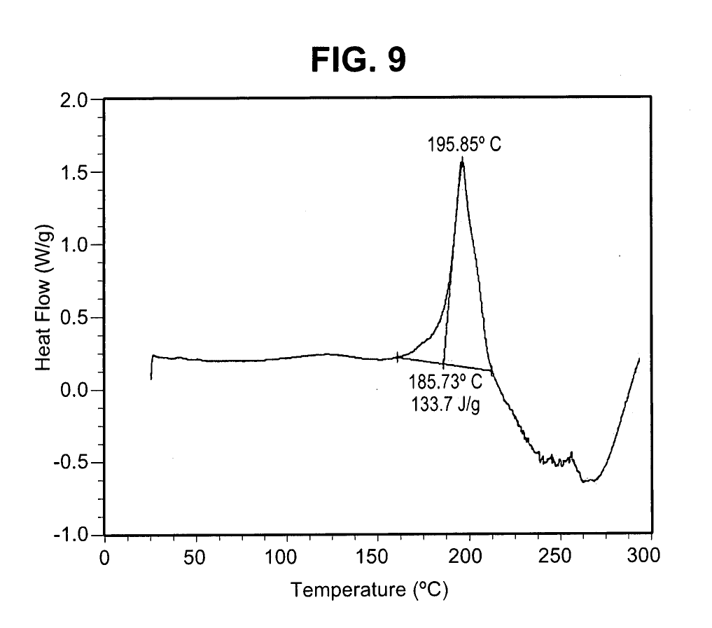

The powder X-ray diffraction (PXRD) pattern of the crystalline sulfate salt of 2-[4-(3-quinolin-6-ylmethyl-3H-[1,2,3]triazolo[4,5-b]pyrazin-5-yl)-pyrazol-1-yl]-ethanol is shown in Table 4. The DSC thermogram for the sulfate salt is shown in FIG. 9.

TABLE 4

| 2θ (°) | Intensity % |
|---|---|
| 8.9 | 31.6 |
| 9.7 | 21.9 |
| 15.2 | 100 |
| 17.1 | 27.5 |
| 18.0 | 94.3 |
| 19.5 | 36.3 |
| 20.1 | 31.2 |
| 22.0 | 56.4 |
| 23.7 | 45.4 |
| 25.0 | 85 |
| 25.7 | 59.8 |
| 27.2 | 73.8 |
| 27.8 | 53.2 |

### Tosylate Salt

The tosylate salt of 2-[4-(3-quinolin-6-ylmethyl-3H-[1,2,3]triazolo[4,5-b]pyrazin-5-yl)-pyrazol-1-yl]-ethanol can be produced with good crystallinity, for example, by stirring 2-[4-(3-quinolin-6-ylmethyl-3H-[1,2,3]triazolo[4,5-b]pyrazin-5-yl)-pyrazol-1-yl]-ethanol with para-toluene sulfonic acid in an appropriate solvent, including but not limited to, including but not limited to, CH₂Cl₂, acetone, THF, acetonitrile, ethyl acetate, methanol, ethanol, water, isopropyl alcohol, or a mixture thereof, then stirring the resulting solid in an appropriate solvent, including but not limited to, including but not limited to, CH₂Cl₂, acetone, THF, acetonitrile, ethyl acetate, methyl tert-butyl ether, methanol, ethanol, water, isopropyl alcohol, or a mixture thereof, followed by crystallization from an appropriate solvent, including but not limited to, including but not limited to, CH₂Cl₂, acetone, THF, acetonitrile, ethyl acetate, methanol, ethanol, water, isopropyl alcohol, or a mixture thereof.

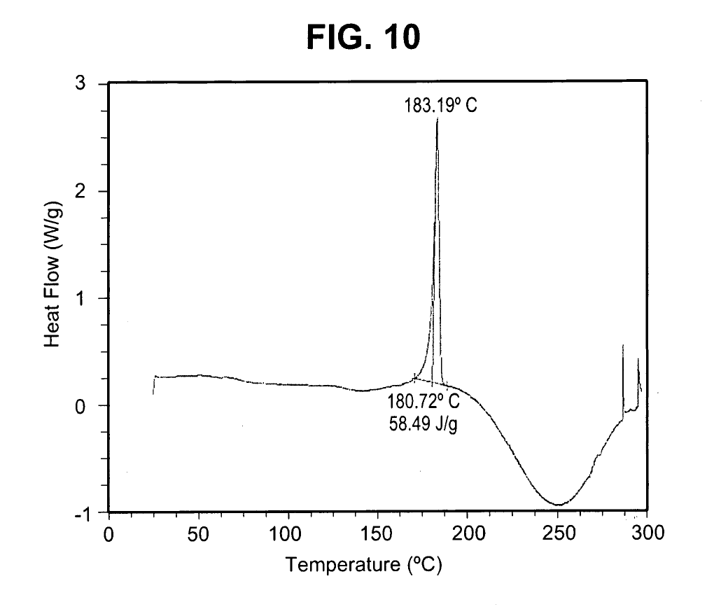

The powder X-ray diffraction (PXRD) pattern of the crystalline tosylate salt of 2-[4-(3-quinolin-6-ylmethyl-3H-[1,2,3]triazolo[4,5-b]pyrazin-5-yl)-pyrazol-1-yl]-ethanol is shown in Table 5. The DSC thermogram for the tosylate salt is shown in FIG. 10.

TABLE 5

| 2θ (°) | Intensity % |
|---|---|
| 10.7 | 49.3 |
| 14.9 | 67.3 |
| 16.5 | 96.9 |
| 17.2 | 80.8 |
| 19.3 | 23.7 |
| 20.3 | 33.9 |
| 22.5 | 28.2 |
| 24.4 | 100 |
| 25.4 | 34.1 |
| 26.6 | 41.5 |
| 27.2 | 47 |
| 28.2 | 32.3 |
| 31.0 | 26 |

The present invention also relates to pharmaceutical compositions comprising the crystalline polymorph salt forms of compound 1 described herein. Pharmaceutical compositions of the present invention may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

### EXAMPLES

The examples and preparations provided below further illustrate and exemplify particular aspects of embodiments of the invention. It is to be understood that the scope of the present invention is not limited in any way by the scope of the following examples.

### Methods and Materials

Salts of PF-04217903 were characterized by their X-ray powder diffraction patterns. Thus, the X-ray powder diffraction patterns of the salts were collected on a Bruker D8 Discover X-ray powder diffractometer with GADDS (General Area Diffraction Detector System) CS operating in reflection mode using Cu Kα radiation (1.54 Å). The tube voltage and amperage were set to 40 kV and 40 mA, respectively. Scans were collected with the sample to detector distance set at 15.0 cm. The samples were scanned for a period of 60 seconds covering a range of 4.5° to 38.7° in 2θ. The diffractometer was calibrated for peak positions in 2θ using a corundum standard. Samples were run in nickel sample holders custom manufactured by Gasser & Sons, Inc (Commack, N.Y.). All analyses were conducted at room temperature, which is generally 20° C.-30° C. Data were collected and integrated using GADDS for WNT software version 4.1.14T. Diffractograms were evaluated using DiffracPlus software, release 2003, with Eva version 9.0.0.2.

To perform an X-ray diffraction measurement on a Bruker D8 Discover X-ray powder diffractometer with GADDS CS used for measurements reported herein, the sample is typically placed into a cavity in the middle of the nickel sample holder. The sample powder is pressed by a glass slide or equivalent to ensure a random surface and proper sample height. The sample holder is then placed into the Bruker instrument and the powder x-ray diffraction pattern is collected using the instrumental parameters specified above. Measurement differences associated with such X-ray powder diffraction analyses result from a variety of factors including: (a) errors in sample preparation (e.g., sample height), (b) instrument errors, (c) calibration errors, (d) operator errors (including those errors present when determining the peak locations), and (e) the nature of the material (e.g. preferred orientation errors). Calibration errors and sample height errors often result in a shift of all the peaks in the same direction. Small differences in sample height when using a flat holder will lead to large displacements in XRPD peak positions. A systematic study showed that a sample height difference of 1 mm could lead to peak shifts as high as 1° 2θ (Chen et al.; J Pharmaceutical and Biomedical Analysis, 2001; 26, 63). These shifts can be identified from the X-ray diffractogram and can be eliminated by compensating for the shift (applying a systematic correction factor to all peak position values) or recalibrating the instrument. As mentioned above, it is possible to rectify differences in measurements from the various instruments by applying a systematic correction factor to bring the peak positions into agreement. In general, this correction factor will bring the measured peak positions into agreement with the expected peak positions and may be in the range of the expected 2θ value±0.2° 2θ. The angle (° 2θ) values and intensity values (as a % of the value of the tallest peak) for each solid form are reported in Tables 1-6.

Differential Scanning calorimetry (DSC) was carried out on a TA Instruments DSC Q1000 V9.1 Build 296. The instrument was calibrated for cell constant and heat capacity using indium and sapphire, respectively. Samples were prepared by weighing 1-3 mg of sample into an aluminum pan which was then covered with a pierced aluminum lid (TA Instruments' part nos. 900786.901 (bottoms) and 900779.901 (top)). Data was analyzed using Universal Analysis 2000 for Windows 2000/XP version 4.3A, Build 4.3.0.6. The experiments started at ambient temperature and heated the sample at 10° C./minute to 350° C. under a nitrogen gas purge (flow rate was 50 ml/min). The thermal events characteristic to each salt are summarized in Table 6.

TABLE 6

| | Transition | |
|---|---|---|
| Salt | onset (° C.) | Proposed Identity of Transition |
| HCl | 214-215 | Decomposition |
| Maleate | 220-221 | Melt |
| Phosphate | 224-225 | Melt/Decomposition |
| Sulfate | 185-186 | Melt/Decomposition |
| Tosylate | 180-181 | Melt/Decomposition |

### Example 1

### Preparation of HCl Salt

29.5 mg of compound 1 was weighed into a 20 mL glass scintillation vial. 1 mL of MeOH was added and solution was stirred. 38.4 μL 2M HCl was pipetted into the solution. Solution capped and stirred and was heated to ˜68° C. in a heater-stirrer module. Heat was turned off and solution continued to stir. Precipitate was observed visually when the solution had cooled to ˜48° C. 500 μL of MeOH was added and solution continued to stir overnight. Solid was recovered using vacuum filtration on a 0.45 μm polytetrafluoroethylene (PTFE) membrane filter. Solid was dried in a 60° C. vacuum oven for ˜30 min.

### Example 2

### Preparation of Maleate Salt

6.95 mg of maleic acid and 22.3 mg of compound 1 were weighed into a 20 mL glass scintillation vial. ˜2 mL of ACN and 20 μL of water was added. Vial was capped and stirred for ˜20 min. Solvent was evaporated by placing under a gentle stream of N₂. ˜3 mL EtOAc and 1 mL IPA was added and solution was capped and stirred overnight. Solid was recovered from solution using vacuum filtration on a 0.45 μm PTFE membrane filter. Solid was dried in a vacuum dessicator for ˜30 min. Solid was placed in a 20 mL glass scintillation vial. ˜2 mL acetone was added and solution was capped and stirred at 50° C. for ˜1 hr. Solid was recovered from solution using vacuum filtration on a 0.45 μm PTFE membrane filter. Solid was dried in a vacuum dessicator for ˜30 min.

### Example 3

### Preparation of Phosphate Salt

21.12 mg of compound 1 was added to a glass screw top HPLC vial. 1 mL of MeOH was pipetted into the vial and it was capped and stirred. 28.357 μL of 2M H₃PO₄ was pipette into the solution. 500 μL MeOH was added and solution was capped and stirred at 60° C. for ˜2 hrs. Heat was removed and solution continued to stir overnight. Solid was observed and was recovered from solution using vacuum filtration on a 0.45 μm PTFE membrane filter. Solid was dried in a 60° C. vacuum oven for 30-60 min. Solid was placed in a 20 mL glass scintillation vial and 5-15 mL IPA was added and solution was capped and stirred overnight. Solid was collected using vacuum filtration on a 0.45 μm PTFE membrane and was dried in a vacuum dessicator for ˜30 min. Solid was placed in a 20 mL glass scintillation vial. ˜10 mL ACN was added and solution was placed in hood and stirred uncapped for ˜48 hrs. Solid was recovered from remaining solution using vacuum filtration on a 0.45 μm nylon filter membrane. Solid was dried in a vacuum dessicator for ˜30 min.

### Example 4

### Preparation of Sulfate Salt

20.92 mg of compound 1 was placed in a glass screw top HPLC vial. 1 mL of MeOH was pipetted into the vial and it was capped and stirred. 28.169 μL of 2M H₂SO₄ was pipetted into the solution. 500 uL of MeOH was then pipetted into the solution. Solution was heated to 60° C. and stirred at this temperature for ˜2 hrs. Heat was removed and solution was stirred overnight. Solid was observed and was recovered using vacuum filtration on a 0.45 μm PTFE membrane filter. Solid was dried in a 60° C. vacuum oven for 30-60 min. Solid was placed in a 20 mL glass scintillation vial. 5-15 mL IPA was added and solution was capped and stirred overnight. Solid was recovered from solution using vacuum filtration on a 0.45 μm PTFE membrane filter and then dried in a vacuum dessicator for ˜30 min. Solid was placed in a 20 mL glass scintillation vial. ˜10 mL ACN was added and solution was capped and stirred overnight. Solid was collected using vacuum filtration on a 0.45 μm nylon filter membrane. Solid was dried for ˜30 min in a vacuum dessicator.

### Example 5

### Preparation of Tosylate Salt

23.79 mg of compound 1 was placed in a glass screw top HPLC vial. 1 mL of MeOH was added and solution was capped and stirred. 31.912 μL of 2M Para-toluene sulfonic acid pipetted into the solution. Solution was stirred at 60° C. for ˜2 hrs. Solution was uncapped and placed under a stream of N₂ until solvent volume was reduced to ˜500 μl. 100 μL aliquots of MTBE was added until precipitation was observed (300 uL was added in total). Solution was capped and stirred and heated to 45° C. and then heat was removed. Solution continued to stir overnight. Solution was transferred to a 20 mL glass scintillation vial. ˜15 mL IPA was added and solution was capped and stirred for ˜72 hrs. Solution was placed under a stream of N₂ until solvent was removed. 5-10 mL of acetone was added. A light brown gel was observed to be stuck to the sides of the glass vial. The solution was transferred to a new 20 mL glass scintillation vial (brownish gum remained in old vial). ˜5 mL acetone and ˜1 mL MeOH was added and solution stirred uncapped in hood. Solid was recovered from solution using vacuum filtration on a 0.45 μm PTFE membrane filter. Solid was dried in a vacuum dessicator for ˜2 hrs.

We claim:

1. A compound comprising a salt selected from the group consisting of 2-[4-(3-quinolin-6-ylmethyl-3H-[1,2,3]triazolo[4,5-b]pyrazin-5-yl)-pyrazol-1-yl]-ethanol hydrochloride salt, 2-[4-(3-quinolin-6-ylmethyl-3H-[1,2,3]triazolo[4,5-b]pyrazin-5-yl)-pyrazol-1-yl]-ethanol maleate salt, 2-[4-(3-quinolin-6-ylmethyl-3H-[1,2,3]triazolo[4,5-b]pyrazin-5-yl)-pyrazol-1-yl]-ethanol phosphate salt, 2-[4-(3-quinolin-6-ylmethyl-3H-[1,2,3]triazolo[4,5-b]pyrazin-5-yl)-pyrazol-1-yl]-ethanol sulfate salt, and 2-[4-(3-quinolin-6-ylmethyl-3H-[1,2,3]triazolo[4,5-b]pyrazin-5-yl)-pyrazol-1-yl]-ethanol tosylate salt.

2. The compound of claim 1, wherein the salt is anhydrous.

3. The compound of claim 1, wherein the salt is a crystalline salt.

4. The compound of claim 1, wherein the salt is a crystalline anhydrous salt.

5. The compound of claim 1, wherein the salt is a substantially pure polymorph.

6. The compound of claim 1, wherein the salt is a compound comprising 2-[4-(3-quinolin-6-ylmethyl-3H-[1,2,3]triazolo[4,5-b]pyrazin-5-yl)-pyrazol-1-yl]-ethanol hydrochloride salt.

7. The compound of claim 1, wherein the salt is a compound comprising a compound comprising 2-[4-(3-quinolin-6-ylmethyl-3H-[1,2,3]triazolo[4,5-b]pyrazin-5-yl)-pyrazol-1-yl]-ethanol maleate salt.

8. The compound of claim 1, wherein the salt is a compound comprising a compound comprising 2-[4-(3-quinolin-6-ylmethyl-3H-[1,2,3]triazolo[4,5-b]pyrazin-5-yl)-pyrazol-1-yl]-ethanol phosphate salt.

9. The compound of claim 1, wherein the salt is a compound comprising a compound comprising 2-[4-(3-quinolin-6-ylmethyl-3H-[1,2,3]triazolo[4,5-b]pyrazin-5-yl)-pyrazol-1-yl]-ethanol sulfate salt.

10. The compound of claim 1, wherein the salt is a compound comprising a compound comprising 2-[4-(3-quinolin-6-ylmethyl-3H-[1,2,3]triazolo[4,5-b]pyrazin-5-yl)-pyrazol-1-yl]-ethanol tosylate salt.

11. The compound of claim 3, wherein the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2 and 20.9±0.2.

12. The compound of claim 3, wherein the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, and 24.8±0.2.

13. The compound of claim 3, wherein the crystalline salt of 2-[4-(3-quinolin-6-ylmethyl-3H-[1,2,3]triazolo[4,5-b]pyrazin-5-yl)-pyrazol-1-yl]-ethanol phosphate has a powder X-ray diffraction pattern comprising peaks at diffraction angles (2θ) of 17.0±0.2, 20.9±0.2, 24.8±0.2, and 25.8±0.2.

14. A pharmaceutical composition comprising the salt claim 1 and a pharmaceutically acceptable carrier.

15. A method of treating abnormal cell growth in a mammal in need of such treatment, the method comprising administering to said mammal a therapeutically effective amount of the salt of claim 1.

16. The method of claim 15, wherein the mammal is a human.

17. The method of claim 15, wherein the abnormal cell growth is mediated by hepatocyte growth factor receptor (c-Met/HGFR) kinase.

18. The method of claim 15, wherein the abnormal cell growth is cancer.

19. The method of claim 18, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

20. The method of claim 18, wherein the cancer is selected from the group consisting of non-small cell lung cancer (NSCLC), squamous cell carcinoma, hormone-refractory prostate cancer, papillary renal cell carcinoma, colorectal adenocarcinoma, neuroblastomas, anaplastic large cell lymphoma (ALCL) and gastric cancer.
